(19) United States
(12) Patent Application Publication
(10) Pub. No.: US 2023/0145397 A1
(43) Pub. Date: May 11, 2023

(54) ION-CONDUCTIVE POLYMERIC MATERIALS AS ELECTROLYTES FOR FUEL CELLS

(71) Applicant: Energao, Inc., Makanda, IL (US)

(72) Inventors: Yong Gao, Makanda, IL (US); Rong Jiang, Makanda, IL (US)

(21) Appl. No.: 17/914,223

(22) PCT Filed: Dec. 21, 2020
(86) PCT No.: PCT/US2020/066360

§ 371 Date: Sep. 23, 2022

Related U.S. Application Data
(60) Provisional application No. 62/993,500, filed on Mar. 23, 2020.

(51) Int. Cl. C08F 14/18; H01M 8/1039; B01D 71/36; B01D 71/32; B01D 71/76; C07C 21/18; C07C 43/176; C07C 39/373; C07F 9/40; C08J 5/22; H01B 1/12

(52) U.S. Cl. CPC C08F 14/185; B01D 71/32; B01D 71/36; B01D 71/76; C07C 21/18; C07C 39/373; C07C 43/176; C07F 9/4071; C08J 5/22; H01B 1/122; H01M 8/1039; H01M 2008/1095

(57) ABSTRACT

Provided in this patent disclosure are two types of novel fluoro-monomers that can be polymerized for the fabrication of ion-exchange fluoropolymers. In addition, new proton-conductive zirconium-perfluorophosphonic acid fluoropolymer membranes that can reduce metal crossovers in redox flow batteries are also provided.

### FIELD OF THE INVENTION

The present disclosure generally relates to (1) novel fluoroalkene monomers and the synthesis of their ion-conductive fluoropolymers, many of which are resistant to free-radical degradations. Such materials can be used in a heavy-duty fuel cell with an enhanced life span; and (2) fluoropolymers and composite fluoropolymers comprising both acid and base groups, which can reduce metal crossovers in redox flow batteries and provide hydration benefits in fuel cells.

### BACKGROUND OF THE INVENTION

Compared with those used in light duty vehicles such as passenger sedans, heavy-duty fuel cells used in trucks, trailers, trains, ships and computer data centers need to possess much enhanced performance and durability. For example, a heavy duty fuel cell-powered trailer needs to be able to handle heavy-duty drive cycles while maintaining its power output over its operation lifetime (e.g., 1,000,000 miles and 30,000 driving hours) in order to be commercially competitive against diesel engine-powered class 8 trucks.

A promising approach to meeting the demands of heavy-duty vehicle applications is higher temperature operation of the fuel cell (100° C. to 120° C.), which can improve the stack efficiency and power output. In comparison, conventional hydrogen fuel cells for passenger sedans are typically operated at a temperature ranging from 60 to 80° C. A fuel cell operating at higher temperatures can also more effectively reject heat, leading to lower cost radiators and increased efficiency through decreased parasitic power loss from cooling. However, operating the fuel cell at temperatures above 100° C. presents challenges for cation-exchange membranes such as Nafion® (e.g., accelerated membrane degradation). Also, higher temperature operation lowers the feasible relative humidity in the fuel cell, resulting in reduced membrane proton conductivity and performance.

Unfortunately, state-of-the-art membrane and ionomer technology is limited in its ability to address the aforementioned issues pertaining to fuel cell heavy-duty applications. Perfluorosulfonic acid (PFSAs) polymers, such as Nafion®, are the state-of-the-art material for the membrane and electrode ionomer for proton exchange membrane fuel cells (PEMFCs). PEMFC conditions cause three types of membrane/ionomer degradation classified as mechanical, chemical, and thermal. Mechanical stresses, associated with swelling and shrinkage of the membrane, are largely addressed using intra-membrane reinforcements. Chemical degradation, however, is more pervasive proceeding by two major pathways initiated by hydroxyl and/or hydroperoxyl radicals formed from hydrogen peroxide produced either as a by-product of the oxygen reduction reaction or reaction of hydrogen and oxygen diffusing through the membrane (M. Zatoń, et al., Sustainable Energy Fuels, 2017, 1, 409-438).

The concentration of H₂O₂ in the membrane and electrode layers largely depends on the operating conditions as well as on the membrane thickness. Relative to those used in light duty vehicles, fuel cell stacks used in heavy duty vehicles require higher efficiencies (higher voltages), longer lifetime, higher temperatures, and lower relative humidity. All of these conditions give rise to significantly higher concentrations of H₂O₂ and radicals. As a consequence, membrane and ionomer degradation become a more serious challenge for the fuel cell systems designed for heavy duty vehicles.

Equally important is the sensitive of the performance of commercial PFSA membranes to humidification. Under low relative humidity conditions (e.g., 20-30% RH), a Nafion® membrane quickly loses water molecules—leading to a significant drop of its proton conductivities. The membrane dehydration problem will become an even more serious challenge at the elevated operating temperatures (100-120° C.) of the heavy duty vehicle application.

On the other hand, PFSA, especially Nafion® membranes are widely used in redox flow batteries such as all vanadium flow batteries. Vanadium cations compete against protons to migrate through a Nafion® membrane from one chamber to another—leading to significant capacity loss during charging and discharging processes.

As a result, there is an urgent need to develop novel fluoropolymer-based cation-exchange membranes and ionomers for heavy-duty PEMFC and redox flow battery applications. In particular, new membrane electrode assemblies comprising free radical-resistant cation-exchange polymers and hydroxyl radical-tolerant catalysts/catalyst supports are needed to meet the year 2030 technical targets set up by the U.S. DOE for heavy duty fuel cells.

In this patent disclosure, we disclose two novel fluoropolymers: (1) a fluoropolymer comprised of mixed fluorocarbon-aromatic hydrocarbon side chains, and (2) a perfluorophosphonic acid (PFPA). The -PO₃H group and aromatic hydrocarbons are tethered to the fluoroalkyl side chains via stable C-P and C-C bonds, respectively, in these polymers. A novel fluoroalkyl free-radical coupling reaction that has been uncovered and extensively studied by our laboratories during recent years was utilized for the synthesis of these new fluoropolymers. Finally, a novel zirconium phosphate composite comprised of PFPAs is used as fuel cell and redox flow battery electrolytes.

### SUMMARY OF THE INVENTION

In one embodiment the present invention provides a fluoroalkene monomer comprising a structure selected from formula (I) or (II).

In the second embodiment the present disclosure provides a proton-conductive fluoropolymer membrane comprising a metal cation, a phosphonate anion of a perfluorophosphonic acid (PFPA) selected from formula (V)-(VIII), and optionally a phosphonate anion of H₂O₃P—R and water.

Other objects and features will be in part apparent and in part pointed out hereinafter.

### DETAILED DESCRIPTION OF THE INVENTION

### A Fluoroalkene Monomer

The first part of the present disclosure is related to a fluoroalkene monomer that is comprised of a polymerizable trifluoroethylene group and a mixed fluorocarbon-hydrocarbon chain. This monomer can be converted into its homopolymer or can be co-polymerized with other monomers such as tetrafluoroethyelene to form a random or block copolymer. The resulted homopolymer or copolymer is comprised of a fluorocarbon-hydrocarbon side chain. The hydrocarbon and fluorocarbon domains in the polymer are covalently bonded via a stable C—C bond.

The fluoroalkene monomer has a general structure selected from formula (I) and (II):

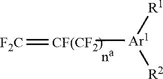

, and

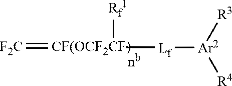

Wherein,
n^{a} is an integer ranging from 1 to 8 and it represents the number of the repeating —CF₂— unit;
n^{b} is an integer number ranging from 0 to 3 and it represents the number of the repeating unit of —OCF₂CR_{f}¹F—;
R_{f}¹ is either F or CF₃;
—L_{f}— is selected from a group of a direct bond, —OCF₂—, —OCF₂CF₂—, —OCF₂CF₂CF₂—, and —OCF₂CF₂CF₂CF₂—;
Ar¹ and Ar² are independently chosen from (C₃-C₂₄)aromatic or heteroaromatic groups;

R¹, R², R³, and R⁴ are independently chosen from a group of H, Cl, Br, I, NO₂, sulfonyl, phosphonyl, CN, OH, amino, a monovalent (C₁-C₁₂)hydrocarbon or fluorocarbon residue, and a bivalent (C₁-C₁₂)hydrocarbon or fluorocarbon residue—two of which taken together can form a cycloalkyl or aromatic ring or a fluorinated aromatic ring.

For the illustrative purposes, only two groups, —R¹ and —R² are attached to Ar¹ and only R³, and R⁴ are bonded to Ar². Those who are skilled in the art will appreciate that it is possible that more substituents can be bonded to the aromatic or heteroaromatic ring of Ar¹ and Ar², respectively. The position of the attachment of R¹, R², R³, and R⁴ will depend on the structure of the Ar¹ and Ar² rings.

In (I), Ar¹ is preferably bonded to the terminal CF₂ group via a C-C covalent bond. In (II), Ar² is also attached to L_{f} or the CF group (if L_{f} is a direct bond) through a covalent bond of C_{—}C. Polymerization of (I) or (II) can give rise to novel hybrid polymers comprised of separate fluorocarbon and hydrocarbon domains.

Without limiting the scope of the present invention, (I-A), (I-B), and (I-C) are used as illustrative examples of formula (I):

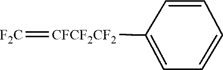

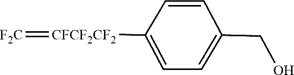

, and

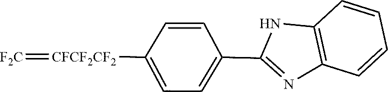

In (I-A), Ar¹ is a phenyl group and R¹ and R² are H. n^{a} is 2.

In (I-B), Ar¹ is a phenylene group. R¹ is CH₂OH and R² is H. n^{a} is 2.

In (I-C), n^{a} is 2; Ar¹ is a p-(2-benzimidazolyl)phenylene group. R¹ and R² are H.

Without limiting the scope of the present invention, (II-A), (II-B), and (II-C) are used as illustrative examples of formula (II):

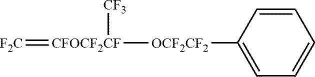

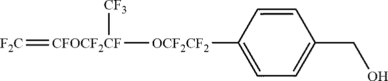

, and

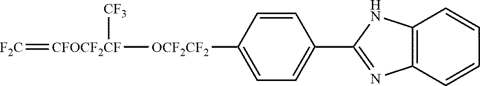

In (II-A), R_{f}¹ is CF₃; n^{b} is 1; Lf is OCF₂CF₂; Ar² is a phenyl group; R³ and R⁴ are H.

In (II-B), R_{f}¹ is CF₃; n^{b} is 1; L_{f} is OCF₂CF₂; Ar² is a phenylene group; R³ is CH₂OH; and R⁴ is H.

In (II-C), R_{f}¹ is CF₃; n^{b} is 1; L_{f} is OCF₂CF₂; Ar² is a p-(2-benzimidazolyl)phenylene group; R³ and R⁴ are H.

In general, the fluoroalkene monomers are synthesized from a fluoroalkyl iodide and an aromatic compound via a novel fluoroalkyl free-radical coupling reaction (A. Bravo, et al., J. Org. Chem., 1997, 62, 7128). Examples of the synthesis of fluoroalkenes are provided with this disclosure. As a result, an aromatic group is covalently bonded to a CF₂ group via a stable C-C bond. For example, in examples (I-B), (I-C), (II-B) and (II-C), a phenylene group is always bonded to CF₂ via a C-C bond. In (I-A) and (II-A), a C-C bond is used for linking a phenyl ring with the fluoroalkyl chain. These C-C covalent bond linkages are of importance to the stability of the polymers that are produced from fluoroalkenes (I) and (II).

In another embodiment, the present disclosure provides an emulsion polymerization method for the synthesis of the homopolymer and copolymer of the fluoroalkene monomers.

Emulsion polymerization is a type of radical polymerization that usually starts with an emulsion incorporating water, monomer, and surfactant. The most common type of emulsion polymerization is an oil-in-water emulsion, in which droplets of monomer (the oil) are emulsified (with surfactants) in a continuous phase of water.

As shown in Examples, water is the solvent and a salt of a fluoroinated carboxylic acid or a fluorinated sulfonic acid is preferably used as the surfactant in our polymerization reaction. The surfactant concentration ranges between 0.0001 wt% and 10 wt% of water. Water-soluble free-radical initiators selected from (NH₄)₂S₂O₈, Na₂S₂O₈, Li₂S₂O₈ and K₂S₂O₈ can be used to promote the polymerization. The free-radical initiator is 0.1 mol% to 10 mol% of the amount of monomers. The surfactant can be any fluoro-surfactants. Examples include ammonium perfluorooctanoate, Capstone™ surfactants, sodium perfluorooctanoate, potassium perfluorooctanoate, and lithium perfluorooctanoate. The surfactant concentration ranges between 0.0001 wt% and 10 wt% of water.

The reaction temperature typically ranges from 25° C. to less than 100° C., and 60° C. is our preferred polymerization temperature. The reaction time can range from one minute to three weeks. However, 1 to 36 hours is our preferred reaction time. Those skilled in the art will appreciate that the emulsion polymerization condition can be fine-tuned according to the structure and purity of the monomer and the equipment available for the polymerization reactions.

Such an emulsion polymerization can also be employed for the synthesis of the copolymer of tetrafluoroethylene and a fluoroalkene monomer disclosed in the present invention. Copolymerization requires the presence of both tetrafluoroethylene and the fluoroalkene monomer together. The molar ratio of these two monomers can vary ranging from 0.01 to 100. Those skilled in the art can appreciate that the desired properties of the copolymer will dictate the molar ratio of these two monomers.

The polymerization for the production of a homopolymer or copolymer can also be carried out in a fluoro-solvent and a fluoroalkyl-based free radical initiator is preferred. Examples of fluoro-solvents include FC-72, HFE-7100, HFE-7500, F-626, CCl₂F₂, CCl₃F, CClF₂H, CCl₂FCCl₂F, CCl₂FCCClF₂ and CClF₂CClF₂. Example of fluoroalkyl-based free radical initiators include 3-P initiator and perfluoro-peroxybenzoic acid. The reaction temperature typically ranges from 25° C. to less than 100° C., and 60° C. is our preferred polymerization temperature. The reaction time can range from one minute to three weeks. However, 1 to 36 hours is our preferred reaction time. For the synthesis of the copolymer of tetrafluoroethylene and the fluoroalkene monomer in this disclose, the molar ratio of the fluoroalkene monomer and tetrafluoroethylene can vary from 0.1 to 10. Those skilled in the art will appreciate that the emulsion polymerization condition can be fine-tuned according to the structure and purity of the monomer and the equipment available for the polymerization reactions.

In the past, numerous literatures (e.g., B. Ameduri, Chem. Eur. J., 2018, 24, 18830) have reported that similar hybrid polymers comprised of hydrocarbon and fluorocarbon domains typically employ esters, amides, sulfonamides, ether, etc. as linkers for tethering two domains together. Unfortunately, esters, amides and sulfonamides are labile to the hydrolytic reactions in water, which will limit the application of these fluorocarbon-hydrocarbon hybrid polymers in electronic devices like fuel cells and redox flow batteries (e.g., C. G. Arges, et al., Proc. Natl. Acad. Sci USA, 2013, 110, 2490). For example, in an all-vanadium redox flow battery, typically an aqueous solution of 4M H₂SO₄ and 1M V⁵⁺ is used as an electrolyte. Under such highly corrosive and oxidative conditions, an acid-labile linker will ensure the low life-span of the polymers in the devices.

As a result, the present invention provides a novel group of hybrid fluoroalkene monomers and thus hybrid polymers that are comprised of covalently C-C bonded fluorocarbon and hydrocarbon domains. These new polymers can have improved stabilities under harsh oxidative and corrosive conditions.

### A Proton-Conductive Fluoropolymer Membrane

A solid membrane like Nafion™ in an electronic device of fuel cells and redox flow batteries is responsible for shuffling protons to the opposite electrode to counter-balance electron charge and discharge. The membrane also separates analyte and catholyte into two separate channels. In addition, many of electrolytes used in the electronic devices are highly corrosive and oxidative (e.g., 4M H₂SO₄ and 1M V⁵⁺). As a result, a solid membrane needs to meet many stringent requirements for its applications in these electronic devices: (1) a water-insoluble solid at room temperature and below 100° C. for PEMFCs; (2) strong mechanic properties to withstand constant shrinking and expanding forces; (3) highly proton-conductive; (4) chemically, thermally and electrochemically stable for long-term applications; (5) low electrolyte crossovers; and (6) low production costs. Unfortunately, very few materials can meet all these requirements. In the past, attempts to increase the proton-conductivity of Nafion™ and other PFSA membranes by increasing the molar ratio of the —SO₃H side chains only led to hygroscopic materials that showed poor mechanic properties. Some of these modified PFSAs have eventually become highly water-soluble.

In the past, attempts have been made to study the use of zirconium phosphate as solid membranes for fuel cell and redox flow battery applications. Zirconium phosphates (also zirconium hydrogen phosphate) are acidic, water-insoluble cation exchange materials that have a layered structure, most of which has a formula of either Zr(HPO₄)₂•H₂O or Zr(PO₄)(H₂PO₄)•2H₂O. Zirconium phosphates have high thermal and chemical stability, solid state ion conductivity, resistance to ionizing radiation, and the capacity to incorporate different types of molecules with different sizes between their layers. There are various phases of zirconium phosphate which vary in their interlaminar spaces and their crystalline structure. Among all the Zirconium phosphate phases the most widely used are the alpha (Zr(HPO₄)₂•H₂O) and the gamma (Zr(PO₄)(H₂PO₄)•2H₂O) phase. Other zirconium phosphate structure such as [alkylamine-H₂]_{1.5}[Zr₃(PO₄)₃F₆] •1.5H₂O was also reported. Zirconium phosphates can have crystal, semi-crystal and amorphous structures.

In comparison, most other zirconium salts do not form such layered structures, which is of importance to the water-solubility, proton conductivity and mechanic properties of zirconium-perfluorophosphonic acid membranes. For example, zirconium sulfate tetrahydrate has a very high water solubility of 52.5 g/100 mL at 25° C. (https://en.wikipedia.org/wiki/Zirconium(IV)_sulfate). In Comparison experiment 1 of Example 13, a mixture of zirconium and —SO₃H— containing Nafion™ failed to form water-insoluble precipitates as the resulted zirconium sulfate derivatives are highly water soluble. High water solubility will not enable zirconium sulfate alone as a solid membrane for the application of fuel cells and redox flow batteries. Attempts to impregnate a solid PFSA membrane like Nafion™ with zirconium sulfate or zirconium oxide also failed the long-term stability tests. For example, in an all-vanadium redox flow battery, an aqueous solution of vanadium in ~4M H₂SO₄ is typically used as an electrolyte. However, zirconium sulfate can leach out of the composite membrane due to the high solubility of zirconium sulfate in water. Zirconium oxides can react with H₂SO₄ to become zirconium sulfate that will leach out of the composite membrane after a short period of uses.

However, zirconium phosphates usually have poor mechanic properties. For example, amorphous zirconium phosphate are powders at 25° C. and it is difficult to form a mechanically strong membrane because of its brittleness. In a PEMFC, due to the variation of the operating temperature and the hydration conditions, a proton-exchange membrane will go through numerous expansion-shrinkage cycles-frequently leading to the formation of cracks and pinholes in the membrane—thus causing the abrupt degradation of the fuel cell performance (R. Borup, et al., Chem Rev., 2007, 107, 3904-3951). As a result, the poor mechanic properties of zirconium phosphates still remain a bottleneck challenge for zirconium phosphates themselves to be used in fuel cells, redox flow batteries and other electronic devices.

One embodiment of the present invention is related to a proton-conductive fluoropolymer membrane comprised of zirconium-perfluorophosphonic acid (PFPA) composite fluoropolymer membrane. The phosphonate groups of a PFPA can form water-insoluble complexes with zirconium cations while the fluorocarbon backbone and side chains of a PFPA can enhance the mechanic properties of the composite by reducing the brittleness that commonly encountered in the metal-organic small molecule composites.

Compared with hydrocarbon-based small molecules or hydrocarbon polymers, fluoropolymers in general have much better chemical, thermal and electrochemical stability. In a C-F bond, the strong electronegativity of a fluorine atom attracts the electron pair shared between the carbon atom and the fluorine atom closer to the fluorine atom-depriving the opportunities of the carbon atom to get oxidized by air oxygen or other oxidants. Also because of the strong electronegativity of fluorine, the non-covalent interactions between two C-F bonds are much stronger than the interactions between two C-C bonds-giving rise to stronger mechanic properties of a fluoropolymer membrane than a hydrocarbon membrane.

One embodiment is related to a proton-conductive fluoropolymer membrane comprising a structural formula of M_{z}(M^{a}ₙO₃P-PRU)ₓ(M^{b}_{n’}O₃P-R)yEₘE’_{m'}•tH₂O, Wherein,
M is a metal cation selected from Zr, Ti, Sn, Ge, Pb, Hf, Ce, Mo and W;
M^{a} and M^{b} are independently selected from H, NH₄, Na, K, Cs, Li, Rb, Mg, Ca, and Sr;
n and n′ are numbers independently selected between 0 and 2;
E and E′ are independently selected from a group of OH, F, (C₁-C₆)hydrocarbon residues, and (C₁-C₆) fluorocarbon residues;
m and m′ are numbers independently chosen between 0 and 6;
t is a number chosen between 0 and 6;
•H₂O represents the structural formula comprising t number of H₂O;
z is an integer selected from a group of 1, 2 and 3;
x is the molar ratio of M^{a}ₙO₃P-PRU over M, which is a number chosen between 0.01 and 3;
y is the molar ratio of M^{b}_{n'}O₃P-R over M, which is a number chosen between 0 and 5;
R is chosen from a group of H, OH, O, Cl, and a monovalent (C₁-C₁₂)hydrocarbon or fluorocarbon residue;
PRU is a phosphine-containing repeating unit (PRU) of a homopolymer or a copolymer and -PRU has a formula selected from (V)-(VIII):

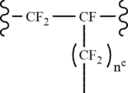

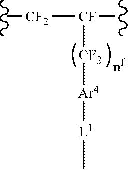

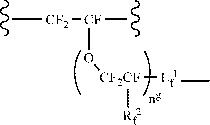

, and

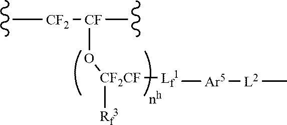

wavy lines indicate the points of attachment to adjacent repeating units of the polymer;
n^{e} and n^{f} are independent integers ranging from 0 to 8;
n^{g} and n^{h} are independent integers ranging from 0 to 3;
R_{f}² and R_{f}³ are independently chosen from F and CF₃;
Ar⁴ and Ar⁵ are independently chosen from (C₃-C₂₄)aromatic or heteroaromatic groups;
L¹ and L² are independently chosen from a group of a direct bond, and a bivalent (C₁-C₁₂)hydrocarbon or fluorocarbon residue;
L_{f}¹ and L_{f}² are independently selected from a group of a direct bond, OCF₂, OCF₂CF₂, OCF₂CF₂CF₂, OCF₂CF₂CF₂CF₂, and OCF₂CF₂CF₂CF₂CF₂.
The composite can be crystal, or semi-crystal or amorphous structures.

Without limiting the scope of the present invention, an example of a proton-conductive fluoropolymer membrane comprising a structural formula of M(M^{a}ₙO₃P-PRU)ₓ(M^{b}_{n'}O₃P-R)_{y}EₘE’_{m'}•tH₂O has a structure of (V-A):

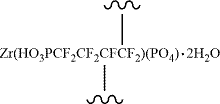

Wherein,
wavy lines indicate the points of attachment to adjacent repeating units of the polymer;
-PRU is (V) wherein n^{e} is 2;
M is Zr;
M^{a} is H;
n is 1;
z is 1;
x is 1;
n′ is 0, which represents that M^{b} is absent from the structure;
m and m′ are 0, which represents the absence of E and E′ in the structure;
R is O;
y is 1;
t is 2. (V-A) contains two H₂O molecules.

In (V-A),

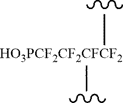

is a derivative of (V) that is a repeating unit of a homopolymer or a copolymer having, for example, the structure of (V-A-1) and (V-A-2), respectively:

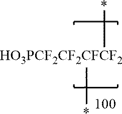

, and

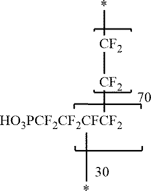

Wherein, * represents the terminus of the polymer backbones; in (V-A-1) the homopolymer contains 100 repeating units while (V-A-2) is a copolymer comprising 70 repeating unit of tetrafluoroethylene and 30 repeating unit of

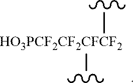

Without limiting the scope of the present invention, another example of a proton-conductive fluoropolymer membrane comprising a structural formula of M(M^{a}ₙO₃P-PRU)ₓ(M^{b}_{n'}O₃P-R)_{y}EₘE’_{m'}•tH₂O has a structure of (VI-A):

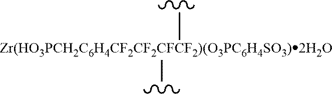

Wherein,
wavy lines indicate the points of attachment to adjacent repeating units of the polymer;
-PRU is (VI)
n^{f} is 2;
Ar⁴ is p-phenylene;
L¹ is CH₂;
M is Zr;
M^{a} is H;
n is 1;
z is 1;
x is 1;
n′ is 0, which represents that M^{b} is absent in the structure;
m and m′ are 0, which represents the absence of E and E′ in the structure;
R is C₆H₄SO₃;
y is 1;
t is 2. (VI-A) contains two H₂O molecules.

Without limiting the scope of the present invention, a further example of a proton-conductive fluoropolymer membrane comprising a structural formula of M(M^{a}ₙO₃P-PRU)ₓ(M^{b}_{n’}O₃P-R)_{y}EₘE’_{m'}•tH₂O has a structure of (VII-A):

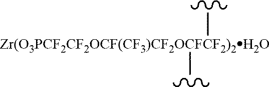

Wherein,
wavy lines indicate the points of attachment to adjacent repeating units of the polymer;
-PRU is (VII)
n^{g} is 1;
L_{f}¹ is OCF₂CF₂;
R_{f}² is CF₃;
M is Zr;
n is 0, which represents the absence of M^{a} from the structure;
z is 1;
x is 2;
m and m′ are 0, which represents the absence of E and E′ in the structure;
y is 0 and M^{b}_{n'}O₃P-R is absent in the structure;
t is 1. (VII-A) contains one H₂O molecules.

A second embodiment of this section is related to a proton-conductive composite fluoropolymer membrane comprising a proton-conductive fluoropolymer membrane and optionally a second fluoropolymer selected from a group of tetrafluoroethylene-perfluoro-3,6-dioxa-4-methyl-7-octenesulfonic acid copolymer, tetrafluoroethylene-perfluoro(5-oxa-6-heptenesulfonic acid) copolymer, tetrafluoroethylene-perfluoro(3-oxa-4-pentenesulfonic acid) copolymer, tetrafluoroethylene-perfluoro-6,9-dioxa-5-methyl-7-undecenesulfonic acid copolymer, tetrafluoroethylene-perfluoro-6-oxa-7-octenesulfonic acid copolymer, and poly(tetrafluoroethylene).

A third embodiment of this section is related to an impregnation method An impregnation method of making a proton-conductive fluoropolymer membrane, comprising
(a) eliminating the solvent from a solution comprising a fluoropolymer of perfluorophosphonic acid (PFPA);
(b) mixing the PFPA residue from (a) with a mixture comprising a metal (4+) salt having a cation selected from Zr⁴⁺, ZrO²⁺, Ti⁴⁺, Sn⁴⁺, Ge⁴⁺, Pb⁴⁺, Hf⁴⁺, Ce⁴⁺, Mo⁴⁺ and W⁴⁺ and an anion, and optionally a phosphonic acid or phosphoric acid in the solvent;
(c) eliminating the solvent from (b);
(d) repeating step (a), (b) and/or (c) if necessary.
Wherein,
the solvent of (a) is selected from water, phosphoric acid, hydrofluoric acid, hydrochloric acid, hydrobromic acid, hydriodic acid, sulfuric acid, nitric acid, organic solvents and the combination thereof. The PFPA concentration in the solution ranges from 0.001 M to 50 M, preferably between 0.1 M and 1 M; the anion of the metal (4+) salt can be any negatively charged ion;

in (b), the metal (4+) salt concentration in the mixture ranges from 0.001 M to 50 M, preferably between 0.1 M and 1 M; the phosphonic acid or phosphoric acid concentration in the mixture ranges from 0.001 M to 50 M, preferably between 0.1 M and 1 M; the solvent is selected from water, phosphoric acid, hydrofluoric acid, hydrochloric acid, hydrobromic acid, hydriodic acid, sulfuric acid, nitric acid, organic solvents and combinations thereof.

A further embodiment of this section is related to a mixing method of making a proton-conductive fluoropolymer membrane, comprising
(a) mixing a fluoropolymer of perfluorophosphonic acid (PFPA), a metal (4+) salt having a cation selected from Zr⁴⁺, Ti⁴⁺, Sn⁴⁺, Ge⁴⁺, Pb⁴⁺, Hf⁴⁺, Ce⁴⁺, Mo⁴⁺ and W⁴⁺ and an anion, and optionally a phosphonic acid or phosphoric acid in a solvent;
(b) eliminating the solvent from the mixture of (b);
(c) repeating step (a), and/or (b) if necessary.
Wherein,
the solvent of (a) is selected from water, phosphoric acid, hydrofluoric acid, hydrochloric acid, hydrobromic acid, hydriodic acid, sulfuric acid, nitric acid, organic solvents and the combination thereof. The PFPA concentration in the solution ranges from 0.001 M to 50 M, preferably between 0.1 M and 1 M; the metal (4+) salt concentration in the mixture ranges from 0.001 M to 50 M, preferably between 0.1 M and 1 M; the anion of the metal (4+) salt can be any negatively charged ion; the phosphonic acid or phosphoric acid concentration in the mixture ranges from 0.001 M to 50 M, preferably between 0.1 M and 1 M.

A final embodiment is related to electronic devices comprising fluoropolymers, or fluoropolymer membranes, or fluoropolymer composite membranes disclosed in the present invention.

Such electronic devices can be fuel cells including hydrogen fuel cells, methanol fuel cells, sodium borohydride fuel cells, ammonia fuel cells and other configurations, redox flow batteries, chlor-alkali cells, electrolyzers, any electric storage systems, any electric generation devices, or water purification devices.

### DEFINITIONS

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

“Aromatic ring” or “aromatic hydrocarbon” or “aromatic group”, abbreviated as “Ar”, refers to a compound that contains a set of covalently bond atoms with the characteristics: (a) a delocalized conjugated π system, most commonly an arrangement of alternating single and double bonds; (b) coplanar structure, with all the contributing atoms in the same plane; (c) contributing atoms arranged in one or more rings; and (d) a total of 4n″ + 2 number of π electrons, where n″=0, 1, 2, 3, etc. Aromatic hydrocarbons can be monocyclic or polycyclic and include heteroaromatic hydrocarbons. Examples of aromatic hydrocarbons include benzene, phenol, aniline, triphenylphosphine, triphenylphosphine oxide, biphenyl, acenaphthene, acenaphthylene, anthracene, fluorene, phenanthren, pyrene, pyridine, imidazole, and naphthalene. “-Ar-” refers to a di- or more-substituted aromatic ring with two substituents at ortho-, meta- or para-positions to each other.

“Heteroaromatic ring” or “heteroaromatic group” or “heteroaromatic compound” refers to an aromatic hydrocarbon having at least one non-carbon atom in the ring. Heteroaromatic hydrocarbons can be monocyclic or polycyclic. Examples of heteroaromatic hydrocarbons include quinoline. In the names of some materials having an aromatic or a heteroaromatic group, x(y) represents a substituent can have a covalent bond to either position x or y of the aromatic or heteroaromatic ring. An example is 3(5)-{1,1,2,2-tetrafluoro-2-[1,2,2-trifluoro-2-(1,2,2-trifluoroethenyloxy)-1-(trifluoromethyl)ethoxy]ethyl}-1,2,4-triazole, wherein 4(5) refers to that the fluoroalkly chain is bonded to either the 3 or 5 position of 1,2,4-triazole.

The rule of “a carbon atom has four covalent bonds; a hydrogen atom has one covalent bond; and an oxygen atom is attached with two covalent bonds” are applied through this invention disclosure. Those skilled in the art can appreciate it that a hydrogen atom may not always be shown in a chemical formula when it is covalently bonded to a carbon atom. A floating bond refers to a covalent bond that is attached to a carbon on a ring provided that the carbon has a total of four covalent bonds including the floating bond.

The term “residue” or “group” refers to two or more atoms bound together as a single unit and forming part of a molecule.

In the present disclosure, “C_{z}” has been used to represent a structure that is comprised of the number (z) of carbon atoms. For example, C₁ refers to a chemical group that has one carbon; C₆ refers to a residue that has six carbons.

The term “monovalent” refers to a chemical group with a valence of one, which thus can form one covalent bond.

The term “bivalent” refers to a chemical group that can form two covalent bonds.

The term “radical”, “free radical” or “free-radical” refers to an uncharged molecule or group (typically highly reactive and short-lived) having an unpaired valence electron.

A “covalent bond” is a chemical bond that involves the sharing of electron pairs between atoms. Examples of “covalent bonds” includes C-C bonds, C-N bonds, and C-O bonds.

“Hydrocarbon” refers to an organic compound that mainly consisting of hydrogen and carbon atoms. Examples include octane, benzene, diethyl ether, aniline, and pyridine.

“Fluorocarbon” or “fluoroalkyl” or “fluorinated” refers to an organic compound derived by replacing all or some of the hydrogen atoms in a hydrocarbon by fluorine atoms (e.g., tetrafluoroethylene).

The term “cycloalkyl” refers to a univalent group formed by removal of one hydrogen atom from a cycloalkane.

The term “cyclofluoroalkyl” refers to a univalent group formed by removal of one hydrogen atom from a cycloalkane that contains at least one fluorine atom.

“Polymer” refers to a compound of high molecular weight derived either by the addition of many smaller molecules, as polyethylene, or by the condensation of many smaller molecules with the elimination of water, alcohol, or the like, as nylon.

“Homopolymer” is a polymer consists of identical monomer units.

The term “copolymer” refers to a polymer made by reaction of two different monomers, with units of more than one kind. It can be either a block and/or a random and/or a sequentially placed copolymer.

A “fluoropolymer” or “fluorocarbon polymer” is a fluorocarbon based polymer with multiple strong carbon-fluorine bonds. Examples include poly(vinyl fluoride), polytetrafluoroethylene, perfluoroalkoxy and poly(chlorotrifluoroethylene).

The term “ion-conductive”, or “ion-exchange”, or “ion exchange”, or “ion conductive”, “ion transportation” or is that an ion can be transported from one site to another. Ionic conduction can lead to an electric current. The SI unit of conductivity is Simens per meter (S/m) and, unless otherwise qualified, it generally refers to 25° C. (standard temperature). The term “electrolyte” refers to a material that transport ions.

The term “anion-conductive” or “anion conductive” refers to the migration of a negatively charged ion from one side to another in a medium.

The term “cation-conductive” or “cation conductive” refers to the migration of a positively charged ion from one side to another in a medium.

The term “proton-conductive” or “proton conductive” refers to the migration of a positively charged proton cation from one side to another in a medium.

The term “solid” refers to a solid state of matter under a temperature ranging from -70 to 200° C.

The term “interaction” or “interactions” refers to any of several forces, especially the ionic bond, hydrogen bond, covalent bond, and metallic bond, by which atoms or ions are bound in a molecule or crystal.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

“Phosphonic acid” is an organophosphorus compound containing C-PO(OH)₂ or C-PO(OM″)₂ groups (wherein M″ = metal or NH₄ cation). “Phosphonyl” refers to a trivalent radical of

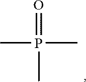

derived from a phosphonic acid group.

“Phosphoric acid” also known as orthophosphoric acid or phosphoric(V) acid, is an acid with the chemical formula H₃PO₄ or PO(OM″)₃ (wherein M″ = metal or NH₄ cation).

“Perfluorophosphonic acid” is a fluoropolymer that has at least one —PO(OH)₂ or —PO(OM″)₂ group (wherein M″ = metal or NH₄ cation) on its side chains. Examples of perfluorophosphonic acid includes tetrafluoroethylene-perfluoro-3,6-dioxa-4-methyl-7-octenephosphonic acid copolymer, tetrafluoroethylene-perfluoro(5-oxa-6-heptenesphosphonic acid) copolymer, 1,1,2,2,3,4,4-heptafluoro-3-butenylphosphonic acid homopolymer, tetrafluoroethylene-1,1,2,2,3,4,4-heptafluoro-3-butenylphosphonic acid copolymer, tetrafluoroethylene-perfluoro-6,9-dioxa-5-methyl-7-undecenephosphonic acid copolymer, tetrafluoroethylene-perfluoro-6-oxa-7-octenephosphonic acid copolymer, and tetrafluoroethylene-perfluoro(3-oxa-4-pentenephosphonic acid) copolymer.

“Sulfonyl” refers to a divalent radical, —SO₂—, derived from a sulfonic acid group.

The term “eliminating the solvent” refers to the removal of the solvent from a solution. Those skilled in the art will appreciate that many methods are available for the removal of the solvent from a solution. Examples include distillation, rotavaporation, freeze-dry, filtration, decanting, and centrifugation.

“Solvent” is a usually liquid substance capable of dissolving or dispersing one or more other substances. Solvents include water and organic solvents. “Organic solvents” are carbon-based substances capable of dissolving or dispersing one or more other substances. Examples of organic solvents include dimethyl sulfoxide (DMSO), N,N-dimethylformamide (DMF), N.N-dimethylacetamide (DMAc), methanol, ethanol, and hexamethylphosphoramide (HMPA).

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

The terms “selected”, “chosen” and “or” refer to make one or more choices including a combination of choices from a number of possibilities.

The terms “between and ” and “from to ” are all inclusive. For example, integer numbers “between 0 and 2” or “from 0 and 2” refer to a group of integer numbers of 0, 1 and 2.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

The following examples illustrate various embodiments of the disclosure. Chemicals and organic solvents mentioned below were purchased from Aldrich (Milwaukee, WI) or Fisher Scientific (Pittsburgh, PA) and used as received. Water was obtained from a Milli-Q water system purchased from Millipore Corporation (Milford, MA). The heavy metal and bacterial contaminant levels in Milli-Q water were below 10 parts per billion. The beads, membranes and aqueous solutions of Nafion® and Aquivion® were purchased from Ion Power, Sigma Aldrich and Solvay Plastics. BT-112 Conductivity Cell from Scrinber Associates (S. Pine, NC) was used for in-plane conductivity tests. CHI6112D Electrochemical Analyzer from CHI Instruments, Inc. (Austin, TX) and UBA5 Battery Analyzer from AA Battery Power Co. (Richmond, CA) were employed for analyzing the fuel cell performance. The in-plane ion conductivity of a membrane was typically measured at 25° C. in Milli-Q water using a four-electrode impedance method (Y. Sone, J. Electrochem. Soc., 1996, 143, 1254). The ionic conductivity (σ) was calculated from current resistance (R) using an equation σ = L/[R•A], wherein A is the cross section area of membrane for resistance measurement and L is the length of two electrodes. Vanadium cation crossover experiments were carried out by adopting the literature protocol (W. Xie, Polymer, 2011, 52, 2032.). The crossover constant, Pₛ is measured at room temperature using a dual chamber U-shape tube. A membrane is mounted between two chambers. One chamber (donor chamber) initially contains an aqueous solution of the salt at a concentration of C_{D}(0) while the other side is initially filled with deionized water with stirring. The concentration of salt in the receptor chamber is monitored as a function of time. The salt permeability is calculated using the following equation: ln[1-2C_{R}(t)/C_{D}(0)][-V//(2A)]=Pₛt.

### Example 1: Synthesis of the Copolymer of Tetrafluoroethylene and Fl, 1,2,2-Tetrafluoro-2-[1,2,2-Trifluoro-2-(1,2,2-Trifluoroethenyloxy)-1-(Trifluoromethyl)Ethoxy]ethyl}-2-Phenyl-Benzimidazole (Polymer 1) and a Composite Solid Membrane of Polymer 1 and Nafion

260 mg Nafion® NR 50 (0.26 mmol) in 15 mL thionyl chloride was refluxed for 24 h. Thionyl chloride was removed and the residue was washed with chloroform 10 mL x 2. The powder in 5 mL dioxane and 15 mL water and 2.52 g sodium sulfite (20 mmol) at 80° C. for 7 days. Removal of the solvents in a vacuum oven at 60° C. with an outside large beaker. The residue was washed with water 15 mL x 3. The polymer was then suspended in 20 mL DMF which was treated with 1.06 g l₂ (10 mmol) at 60° C. (shielding the lights). After 24 h, the polymer was filtered and the polymer was washed with 10 mLx 3 1M NaHSO₃ (sodium bisulfite) and saturated NaHCO₃ and water 10 mL x 2.

In a round bottom flask with a condenser, -189 mg polymer above in 5 mL DMSO was mixed with 100 mg (0.39 mmol) 2-phenyl-benzimidazole, 25 mg (14 µL) H₂SO₄ and 22 mg (0.08 mmol) FeSO₄. Then, 35% H₂O₂ (0.1 mL, 100 µL) was added. At room temperature for 4 days. Water was added to precipitate the polymer (decant or centrifugation). The polymer was washed with water 15 mL x 3, then washed with acetone 10 mL x 2 to yield Polymer 1. ¹H NMR (400 MHz, DMSO-d6): δ 8.82-7.20 (9H, m).

Preparation of a solid membrane of Polymer 1. 200 mg of Polymer was dissolved in 10 mL DMSO. The solvent was removed in vacuo. The membrane was boiled with 20 mL 1M H₂SO₄ for 1 h. The solution was cooled down to ambient temperature. Then, water was decanted and the film was washed with water until pH 7.0. The membrane was boiled in in 20 mL water 1 h. Cooled down to room temperature. The membrane has many pinholes and its proton conductivity in Milli-Q water at 25° C. was 1.63 mS/cm.

Preparation of a solid membrane comprising 10 wt% Polymer 1and 90 wt% Nafion®. 20 mg of Polymer 1 and 180 mg Nafion® NR 50 was dissolved in 10 mL DMSO. The solvent was removed in vacuo. The membrane was boiled with 20 mL 1M H₂SO₄ for 1 h. The solution was cooled down to ambient temperature. Then, water was decanted and the film was washed with water until pH 7.0. The membrane was boiled in in 20 mL water 1 h. Cooled down to room temperature. The membrane has a proton conductivity of 33 mS/cm.

Preparation of a solid membrane comprising 30 wt% Polymer 1 and 70 wt% Nafion®. 60 mg of Polymer 1 and 140 mg Nafion® NR 50 was dissolved in 10 mL DMSO. The solvent was removed in vacuo. The membrane was boiled with 20 mL 1M H₂SO₄ for 1 h. The solution was cooled down to ambient temperature. Then, water was decanted and the film was washed with water until pH 7.0. The membrane was boiled in in 20 mL water 1 h. Cooled down to room temperature. The membrane has a proton conductivity of 19 mS/cm.

### Example 2: Synthesis of 1,1,2,3,3,4,4-Heptafluoro-4-Phenyl-1-Butene

To a mixture of 1,2-Dichloro-4-iodoperfluorobutane (1 mmol), benzene (3 mmol), 98 mg H₂SO₄ and 83 mg (0.08 mmol) FeSO₄7H₂O in 5 mL DMSO. Then, 35% H₂O₂ (0.2 mL) was added. After 4 days at room temperature, DMSO was evaporated in vacuo. The residue was washed with water 10 mL x 2 and then was further purified by flash chromatography (30% EtOAc in hexane) to yield 1,2-dichloro-4-phenylperfluorobutane (∼31%). ¹H NMR (400 MHz, CDCl₃) δ 7.6-7.2 (m, 5 H).

1,2-dichloro-4-phenylperfluorobutane (0.2 mmol) was added to a suspension of zinc dust (1 mmol) and zinc chloride (0.1 mmol) in 10 mL EtOH. After refluxing for over 4 days, the solvent was removed in vacuo. The residue was extracted with chloroform 10 mL x 3 and EtOAc 10 mL x 2. The organic extracts were combined and the solvent was removed. The residue was further purified by flash chromatography (30% EtOAc in hexane) to yield 1,1,2,3,3,4,4-Heptafluoro-4-phenyl-1-butene (~75%). ¹H NMR (400 MHz, CDCl₃) δ 7.8-7.27 (m, 5 H); MS m/z 259.14 (80, [M+H]⁺).

### Example 3: Polymerization and Sulfonation of 1,1,2,3,3,4,4-Heptafluoro-4-Phenyl-1-Butene (Polymer 2)

In a round-bottom flask, 20 mL water was degassed with nitrogen for 30 min. Then, 15 mg ammonium perfluorooctanoate, 160 mg sodium phosphate dibasic and 50 mg sodium bisulfite were added. After 10 min, 500 mg 1,1,2,3,3,4,4-heptafluoro-4-phenyl-1-butene was introduced. After another 10 min, the solution was frozen to -78° C. under high vacuum. After warmed to room temperature under argon, 25 mg potassium persulfate was added. The mixture was stirred at 60° C. for 3 days under argon. The polymer was precipitated out. After filtration, the polymer residue was washed with water 10 mL x 3 followed by acetone 10 mL x 2 to yield 420 mg poly(1,1,2,3,3,4,4-heptafluoro-4-phenyl-1-butene) (Polymer 2). ¹H NMR (400 MHz, DMSO-d6) δ 7.7-7.3 (m, 5 H).

50 mg poly(1,1,2,3,3,4,4-heptafluoro-4-phenyl-1-butene) was treated with a mixture of 10 mL concentrated sulfuric acid and 1 mL chlorosulfonic acid under an ice bath. After 12 h, the polymer was removed from the sulfuric acid mixture. However, the sulfonated poly(1,1,2,3,3,4,4-heptafluoro-4-phenyl-1-butene) is highly water-soluble and attempts to cast a membrane failed.

### Example 4: Synthesis of 8-phenyl-perfluoro-3,6-dioxa-5-methyl-octene

To a mixture of 1,2-dichloro-1,1,2,4,4,5,7,7,8,8-decafluoro-5-trifluoromethyl-8-iodo-3,6-dioxaoctane (S. D. Pedersen, J. Org. Chem. 1996, 61, 8024) (1 mmol) benzene (3 mmol), 98 mg H₂SO₄ and 83 mg (0.08 mmol) FeSO₄7H₂O in 5 mL DMSO. Then, 35% H₂O₂ (0.2 mL) was added. After 4 days at room temperature, DMSO was evaporated in vacuo. The residue was washed with water 10 mL x 2 and then was further purified by flash chromatography (30% EtOAc in hexane). Then, the residue was added to a suspension of zinc dust (5 mmol) and zinc chloride (0.5 mmol) in 30 mL EtOH. After refluxing for over 4 days, the solvent was removed in vacuo. The residue was extracted with chloroform 10 mL x 3 and EtOAc 10 mL x 2. The organic extracts were combined and the solvent was removed. The residue was further purified by flash chromatography (30% EtOAc in hexane) to yield 8-phenyl-perfluoro-3,6-dioxa-5-methyl-octene (14%). ¹H NMR (400 MHz, CDCl₃) δ 7.9-7.3 (m, 5 H).

### Example 5: Synthesis of P-(1,1,2,2,3,4,4-Heptafluoro-3-Butenyl)Benzyl Alcohol

To a mixture of 1,2-Dichloro-4-iodoperfluorobutane (1 mmol), benzyl alcohol (2 mmol), 98 mg H₂SO₄ and 83 mg (0.08 mmol) FeSO₄7H₂O in 5 mL DMSO. Then, 35% H₂O₂ (0.2 mL) was added. After 4 days at room temperature, DMSO was evaporated in vacuo. The residue was washed with water 10 mL x 2 and then was further purified by flash chromatography (30% EtOAc in hexane) to yield 1,2-dichloro-perfluorobut-3-enyl-benzyl alcohol (127 mg).

1,2-Dichloro-perfluorobut-3-enyl-benzyl alcohol (0.2 mmol) was added to a suspension of zinc dust (1 mmol) and zinc chloride (0.1 mmol) in 10 mL EtOH. After refluxing for over 4 days, the solvent was removed in vacuo. The residue was extracted with chloroform 10 mL x 3 and EtOAc 10 mL x 2. The organic extracts were combined and the solvent was removed. The residue was further purified by flash chromatography (30% EtOAc in hexane) to yield p-(1,1,2,2,3,4,4-heptafluoro-3-butenyl)benzyl alcohol (90%). ¹H NMR (400 MHz, CDCl₃) δ 7.7-7.2 (m, 4 H), 4.7 (s, 2 H); MS m/z 289.12 (100, [M+H]⁺).

### Example 6: Polymerization of P-(1,1,2,2,3,4,4-Heptafluoro-3-Butenyl)Benzyl Alcohol (Polymer 3)

In a round-bottom flask, 20 mL water was degassed with nitrogen for 30 min. Then, 15 mg ammonium perfluorooctanoate, 160 mg sodium phosphate dibasic and 50 mg sodium bisulfite were added. After 10 min, 500 mg p-(1,1,2,2,3,4,4-heptafluoro-3-butenyl)benzyl alcohol was introduced. After another 10 min, the solution was frozen to -78° C. under high vacuum. After warmed to room temperature under argon, 25 mg potassium persulfate was added. The mixture was stirred at 60° C. for 3 days under argon. The polymer was precipitated out. After filtration, the polymer residue was washed with water 10 mL x 3 followed by acetone 10 mL x 2 to yield 420 mg poly(1,1,2,3,3,4,4-heptafluoro-4-phenyl-1-butene) (Polymer 3). ¹H NMR (400 MHz, DMSO-d6) δ 7.7-7.2 (m, 4 H), 4.9 (s, 2 H).

### Example 7: Synthesis of 1,1,2,3,3,4,4-Heptafluoro-4-(Diethoxyphosphinoyl)-1-Butene

To a mixture of 1,2-Dichloro-4-iodoperfluorobutane (1 mmol), tetraethyl pyrophosphite (2 mmol), 98 mg H₂SO₄ and 83 mg (0.08 mmol) FeSO₄7H₂O in 5 mL DMSO. Then, 35% H₂O₂ (0.2 mL) was added. After 4 days at room temperature, DMSO was evaporated in vacuo. The residue was washed with water 10 mL x 2 and then was further purified by flash chromatography (30% EtOAc in hexane) to yield 1-(3,4-dichloro-1,1,2,2,3,4,4-heptafluorobutyl)-diethyl-phosphonate (∼30%). ¹H NMR (400 MHz, CDCl₃) δ 4.27 (m, 4 H), 1.38 (m, 6 H).

1-(3,4-dichloro-1,1,2,2,3,4,4-heptafluorobutyl)-diethoxyphosphine (0.2 mmol) was added to a suspension of zinc dust (1 mmol) and zinc chloride (0.1 mmol) in 10 mL EtOH. After refluxing for over 4 days, the solvent was removed in vacuo. The residue was extracted with chloroform 10 mL x 3 and EtOAc 10 mL x 2. The organic extracts were combined and the solvent was removed. The residue was further treated with 10 mL 30% hydrogen peroxide in 10 mL EtOH. After 12 h, the solvent was removed in vacuo and the residue was purified by flash chromatography (30% EtOAc in hexane) to yield 1,1,2,3,3,4,4-Heptafluoro-4-(diethoxyphosphinoyl)-1-butene. ¹H NMR (400 MHz, CDCl₃) δ 4.28 (m, 4 H), 1.38 (m, 6 H). GC-MS m/z 482.02 (100, M⁺).

### Example 8: Polymerization of 1,1,2,3,3,4,4-Heptafluoro-4-(diethoxyphosphinoyl)-1-Butene (Polymer 4)

In a round-bottom flask, 20 mL water was degassed with nitrogen for 30 min. Then, 15 mg ammonium perfluorooctanoate, 160 mg sodium phosphate dibasic and 50 mg sodium bisulfite were added. After 10 min, 500 mg p-(1,1,2,2,3,4,4-heptafluoro-3-butenyl)benzyl alcohol was introduced. After another 10 min, the solution was frozen to -78° C. under high vacuum. After warmed to room temperature under argon, 25 mg potassium persulfate was added. The mixture was stirred at 60° C. for 3 days under argon. The polymer was precipitated out. After filtration, the polymer residue was washed with water 10 mL x 3 followed by acetone 10 mL x 2 to yield 420 mg poly(1,1,2,3,3,4,4-heptafluoro-4-(diethoxyphosphinoyl)-1-butene) (Polymer 4). ¹H NMR (400 MHz, DMSO-d6) δ 4.25 (m, 4 H), 1.36 (m, 6 H).

### Example 9: Synthesis of the Homopolymer of 1,1,2,2,3,4,4-Heptafluoro-3-Butenylphosphonic Acid (Polymer 5)

100 mg poly(1,1,2,3,3,4,4-heptafluoro-4-(diethoxyphosphinoyl)-1-butene) (Polymer 4) in 10 mL DMSO was treated with 45 mg trimethylsilyl chloride and 56 mg lithium iodide. After 4 days at 50° C., DMSO was removed in vacuo to yield 92 mg homopolymer of 1,1,2,2,3,4,4-heptafluoro-3-butenylphosphonic acid (Polymer 5). ¹H NMR (400 MHz, DMSO-d6): no proton signal detected.

### Example 10: Synthesis of the Homopolymer Of P-(1,1,2,2,3,4,4-Heptafluoro-3-Butenyl)Benzyl-Phosphonic Acid (Polymer 6)

To a mixture of 100 mg polymer (synthesized from example 4) in 10 mL dry toluene, 168 mg (0.525 mmol) Znl₂ was added with 175 mg (1.05 mmol) triethyl phosphite. The mixture was stirred at reflux (-120° C.) for 2 days. Then, the solvent was removed in vacuo. The residue was dissolved in 15 mL chloroform, which was washed with 1 M HCl 10 mL and then water 10 mL x 2 to yield poly(p-(1,1,2,2,3,4,4-heptafluoro-3-butenyl)benzylphosphonic acid diethyl ester). ¹H NMR (400 MHz, DMSO-d6) δ 7.9-7.2 (m, 4 H), 5.4 (m, 2 H), 4.2 (m, 4 H), 1.38 (m, 6 H).

100 mg poly(1,1,2,3,3,4,4-heptafluoro-4-(diethoxyphosphinoyl)-1-butene) in 10 mL DMSO was treated with 45 mg trimethylsilyl chloride and 56 mg lithium iodide. After 4 days at 50° C., DMSO was removed in vacuo to yield 92 mg homopolymer of p-(1,1,2,2,3,4,4-heptafluoro-3-butenyl)benzylphosphonic acid (Polymer 6). ¹H NMR (400 MHz, DMSO-d6) δ 7.9-7.2 (m, 4 H), 5.3 (m, 2 H).

### Example 11: Synthesis of the Copolymer of Tetrafluoroethylene and {1,1,2,2-Tetrafluoro-2-[1,2,2-Trifluoro-2-(1,2,2-Ttrifluoroethenyloxy)-1-(Trifluoromethyl)Ethoxy]ethyl}Phosphonic Acid (Polymer 7)

260 mg Nafion® NR 50 (0.26 mmol) in 15 mL thionyl chloride was refluxed for 24 h. Thionyl chloride was removed and the residue was washed with chloroform 10 mL x 2. The powder in 5 mL dioxane and 15 mL water and 2.52 g sodium sulfite (20 mmol) at 80° C. for 7 days. Removal of the solvents in a vacuum oven at 60° C. with an outside large beaker. The residue was washed with water 15 mL x 3. The polymer was then suspended in 20 mL DMF which was treated with 1.06 g l₂ (10 mmol) at 60° C. (shielding the lights). After 24 h, the polymer was filtered and the polymer was washed with 10 mLx 3 1M NaHSO₃ (sodium bisulfite) and saturated NaHCO₃ and water 10 mL x 2.

800 mg the aforementioned polymer in 20 mL of DMSO was treated with 465 mg tetraethyl pyrophosphite, 116 mg H₂SO₄ and 102 mg FeSO₄. Then, 35% H₂O₂ (0.46 mL, 460 µL) was added. After 3 days at room temperature, water was added to precipitate the polymer. The polymer was washed with water 15 mL x 3, then washed with acetone 10 mL x 2.

100 mg poly(1,1,2,3,3,4,4-heptafluoro-4-(diethoxyphosphinoyl)-1-butene) in 10 mL DMSO was treated with 45 mg trimethylsilyl chloride and 56 mg lithium iodide. After 4 days at 50° C., DMSO was removed in vacuo to yield 70 mg the copolymer of tetrafluoroethylene and {1,1,2,2-tetrafluoro-2-[1,2,2-trifluoro-2-(1,2,2-trifluoroethenyloxy)-1-(trifluoromethyl)ethoxy]ethyl}phosphonic acid (Polymer 7). ¹H NMR (400 MHz, DMSO-d6): no proton signals detected.

### Example 12: Preparation of Proton-Conductive Solid Membranes

Membrane 1: a mixture of polymer 6 (0.248 mmol of phosphine) in 10 mL water was treated with 40 mg zirconyl chloride octahydrate (ZrOCl8H₂O, 0.124 mmol). After stirring for 10 min at room temperature, the solution became cloudy and after 2 h, solids precipitated out at 80° C. The membrane was washed with water 20 mL x 5 and it was boiled in distilled water for 2 h. The membrane was then hot pressed at 145° C. under 9 tons for 2 h and a solid membrane with strong mechanic properties was obtained. SEM-EDS analysis confirmed the composition of Zr, P, F and C in membrane 1. For a 98 µm thick film, Young’s modulus (25° C., 25% RH): 158 MPa; Elongation at break: 190%; in-plane proton conductivity (25° C., in distilled water): σ=10 mS/cm. The VO²⁺ crossover constant Pₛ = 3x10⁻⁸ (cm²/min). The commercial Nafion™ has a Pₛ value of 6.7x10⁻⁷ (cm²/min).

Membrane 2: a mixture of polymer 6 (0.124 mmol of phosphine) and phosphoric acid (0.124 mmol) in 10 mL water was treated with 40 mg zirconyl chloride octahydrate (ZrOCl8H₂O, 0.124 mmol). After stirring for 10 min at room temperature, the solution became cloudy and after 2 h at 80° C., solids precipitated out. Then, the dish was heated to 80° C. in an oven. The membrane was washed with water 20 mL x 5 and it was boiled in distilled water for 2 h. The membrane was then hot pressed at 145° C. under 9 tons for 2 h and a solid membrane with strong mechanic properties was obtained. SEM-EDS analysis confirmed the composition of Zr, P, F and C in membrane 2. For a 80 µm thick film, Young’s modulus (25° C., 25% RH): 72 MPa; Elongation at break: 110%; in-plane proton conductivity (25° C., in distilled water): σ=15 mS/cm. The VO²⁺ crossover constant Pₛ = 6x10⁻⁸ (cm²/min).

Membrane 3: a mixture of polymer 6 (0.124 mmol of phosphine) and m-sulfonylbenzenephophonic acid (0.124 mmol) in 10 mL water was treated with 40 mg zirconyl chloride octahydrate (ZrOCl8H₂O, 0.124 mmol). After stirring for 10 min, the solution became cloudy and the mixture was poured to the NaOH-treated Nafion film (N-211) in a dish. After 2 h at 80° C., solids precipitated out on the top of the N-211 film. The composite membrane was then hot pressed at 145° C. under 9 tons for 2 h. A solid membrane with strong mechanic properties was obtained. Then, the membrane was washed with water 20 mL x 5 and it was boiled in distilled water for 2 h. TEM-EDS analysis confirmed the composition of Zr, P, F and C. SEM-EDS analysis confirmed the composition of Zr, P, F and C in membrane 3. For a 110 µm thick film, Young’s modulus (25° C., 25% RH): 257 MPa; Elongation at break: 237%; in-plane proton conductivity (25° C., in distilled water): σ=68 mS/cm. The VO²⁺ crossover constant Pₛ = 3x10⁻⁸ (cm²/min).

Membrane 4: a mixture of polymer 5 (0.248 mmol of phosphine) in 10 mL water was treated with 40 mg zirconyl chloride octahydrate (ZrOCl8H₂O, 0.124 mmol). After stirring for 10 min at room temperature, the solution became cloudy and after 2 h at 80° C., solids precipitated out. The membrane was washed with water 20 mL x 5 and it was boiled in distilled water for 2 h. The membrane was then hot pressed at 145° C. under 9 tons for 2 h and a solid membrane with strong mechanic properties was obtained. TEM-EDS analysis confirmed the composition of Zr, P, F and C in membrane 4. SEM-EDS analysis confirmed the composition of Zr, P, F and C. For a 77 µm thick film, Young’s modulus (25° C., 25% RH): 167 MPa; Elongation at break: 203%; in-plane proton conductivity (25° C., in distilled water): σ=7 mS/cm. The VO²⁺ crossover constant Pₛ = 4x10⁻⁸ (cm²/min).

Membrane 5: a mixture of polymer 7 (0.124 mmol of phosphine) and phosphoric acid (0.124 mmol) in 10 mL water was treated with 40 mg zirconyl chloride octahydrate (ZrOCl8H₂O, 0.124 mmol). After stirring for 10 min at room temperature, the solution became cloudy and after 2 h at 80° C., solids precipitated out. The membrane was washed with water 20 mL x 5 and it was boiled in distilled water for 2 h. The membrane was then hot pressed at 145° C. under 9 tons for 2 h and a solid membrane with strong mechanic properties was obtained. TEM-EDS analysis confirmed the composition of Zr, P, F and C in membrane 5.

### Example 13: Comparison Experiments on the Preparation of Proton-Conductive Solid Membranes

Comparison experiment 1: a mixture of a water-propanol solution of Nafion™ (0.248 mmol of -SO₃H) in 10 mL water was treated with 40 mg zirconyl chloride octahydrate (ZrOCl8H₂O, 0.124 mmol). After stirring for 10 min at room temperature, the mixture was still transparent and no cloudiness was observed. Then, after 7 days at 80° C., no precipitations were found. The mixture remained clear and no film was formed.

Comparison experiment 2: a mixture of phosphoric acid (0.248 mmol) in 10 mL water was treated with 40 mg zirconyl chloride octahydrate (ZrOCl8H₂O, 0.124 mmol). After stirring for 10 min at room temperature, the solution became cloudy. After 2 h at 80° C., white solids precipitated out. The resulted solids were washed with water 20 mL x 5. However, no sturdy film was formed. The solids were very brittle and under 1 ton of pressure, it became many small pieces. No mechanic stress tests, proton conductivity tests and metal cation crossover experiments were able to be conducted due to its poor mechanic properties.

Comparison experiment 3: to a solution of N,N-bis(phosphonomethyl)glycine (1 mmol) in 10 mL water was added a mixture of zirconyl chloride octahydrate (ZrOCl8H₂O, 0.67 mmol) in 2 mL 3 M hydrofluoric acid. After 2 h at 80° C., solids precipitated out. The membrane was washed with water 20 mL x 5 and it was boiled in distilled water for 2 h. The membrane was then hot pressed at 145° C. under 9 tons for 2 h and pellets were obtained. Young’s modulus (25° C., 16% RH): 12 MPa; Elongation at break: 4%; in-plane proton conductivity (25° C., in distilled water): σ=0.1 mS/cm. SEM-EDS analysis confirmed the composition of Zr, P, and C in this membrane.

### Example 14: All-Vanadium Redox Flow Battery Test

A 5 cm² flow battery comprising Membrane 3 was used in our test. The negative side was comprised of 1 M V²⁺/V³⁺ in 4 M H₂SO₄ and the positive chamber has 1 M VO²⁺/V⁵⁺ in 4 M H₂SO₄. The flow rates are ~10 mL/min for both sides. Three laser-perforated carbon papers (370 µm thick, activated at 400° C. for 30 h before use) were utilized on each electrodes. A maximum discharge power density of 108 mW/cm² was achieved at a state of charge (SOC) of over 90%.

### Example 15: H₂/O₂ Fuel Cell Test

A 5 cm² flow battery comprising Membrane 3 was used in our test. Both anode and cathode contained 0.4 mg/cm² Pt/C and 20 wt% Nafion™ while cathode. H₂ was used to feed anode with a flow rate of 50 ccm and a back pressure of 20 psi. For the cathode side, oxygen streams were employed as the oxidative fuel. The oxygen flow rate was 70 ccm at 80° C. and the back pressure in 20 psi. The fuel cell fixture was maintained during the experiments. A maximum power output of 321 mW/cm² was obtained at a current of 625 mA/cm².

1. A fluoroalkene monomer comprising a structure of formula selected from (I) or (II): 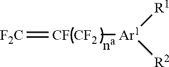 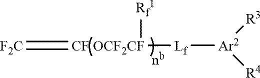
wherein,
n^{a} represents the number of the repeating -CF₂- units and is an integer from 1 to 8;
n^{b} is an integer ranging from 0 to 3;
R_{f}¹ is either F or CF₃;
-Lf- is selected from the group consisting of a direct bond, -OCF₂-, -OCF₂CF₂-, -OCF₂CF₂CF₂-, -OCF₂CF₂CF₂CF₂-, and -OCF₂CF₂CF₂CF₂CF₂-.
Ar¹ and Ar² are independently chosen from (C₃-C₂₄)aromatic or heteroaromatic groups; and
R¹, R², R³, and R⁴ are independently selected from the group consisting of H, Cl, Br, I, NO₂, sulfonyl, phosphonyl, CN, OH, amino, a monovalent (C₁-C₁₂ₒ)hydrocarbon or fluorocarbon residue, and a bivalent (C₁-C₁₂)hydrocarbon or fluorocarbon residue, wherein two of which taken together can form a cycloalkyl or aromatic ring or a fluorinated aromatic ring.

2. A fluoroalkene monomer according to claim 1, wherein n^{a} is from 2 to 8; n^{b} is from 0 to 3; R_{f}¹ is CF₃; -Lf- is selected from the group consisting of -OCF₂-, -OCF₂CF₂-, -OCF₂CF₂CF₂-, -OCF₂CF₂CF₂CF₂-, and -OCF₂CF₂CF₂CF₂CF₂-; Ar¹ and Ar² are independently chosen from (C₃-C₁₆)aromatic or heteroaromatic groups; and R¹, R², R³, and R⁴ are independently selected from the group consisting of H, Cl, Br, I, NO₂, CN, OH, amino, a monovalent (C₁-C₁₀)hydrocarbon or fluorocarbon residue, and a bivalent (C₁-C₁₀)hydrocarbon or fluorocarbon residue, wherein two of which taken together can form a cycloalkyl or aromatic ring or a fluorinated aromatic ring.

3. A fluoroalkene monomer according to claim 2, wherein n^{a} is 2; n^{b} is from 0 to 2; R_{f}¹ is CF₃; -Lf- is selected from the group consisting of -OCF₂CF₂-, -OCF₂CF₂CF₂CF₂-, and -OCF₂CF₂CF₂CF₂CF₂-; Ar¹ and Ar² are independently chosen from (C₆-C₁₄)aromatic or heteroaromatic groups; and R¹, R², R³, and R⁴ are independently selected from the group consisting of H, a monovalent (C₁-C₆)hydrocarbon or fluorocarbon residue, and a bivalent (C₁-C₆)hydrocarbon or fluorocarbon residue, wherein two of which taken together can form a cycloalkyl or aromatic ring or a fluorinated aromatic ring.

4. A fluoroalkene monomer according to claim 3, wherein n^{a} is 2; n^{b} is 1; R_{f}¹ is CF₃; -Lf- is -OCF₂CF₂-; Ar¹ and Ar² are independently chosen from (C₆-C₁₄)aromatic or heteroaromatic groups; and R¹, R², R³, and R⁴ are independently selected from the group consisting of H, a monovalent C₁-C₆ hydrocarbon residue, and a bivalent C₁-C₆ hydrocarbon residue.

5. A fluoroalkene monomer according to claim 4, wherein n^{a} is 2; n^{b} is 1; R_{f}¹ is CF₃; -Lf- is -OCF₂CF₂-; Ar¹ and Ar² are p-phenylene; R¹ and R³ are H; and R² and R⁴ are CH₂OH.

6. A fluoroalkene monomer according to claim 4, wherein n^{a} is 2; n^{b} is 1; R_{f}¹ is CF₃; -Lf- is -OCF₂CF₂-; Ar¹ and Ar² are p-phenylene; and R¹, R², R³ and R⁴ are H.

7. A homopolymer of a fluoroalkene monomer according to claim 1 .

8. A copolymer of tetrafluoroethylene and a fluoroalkene monomer according to claim 1 .

9. A method of forming a homopolymer from a
fluoroalkene monomer according to claim 1 , which comprises polymerizing said fluoroalkene monomer in a perfluorinated solvent at a reaction temperature with a perfluorinated free radical initiator, and optionally under a ultraviolet light;
wherein,
the perfluorinated solvent is a fluorinated organic solvent selected from the group consisting of FC-72, HFE-7100, HFE-7500, F-626, CCl₂F₂, CCl₃F, CClF₂H, CCl₂FCCl₂F, CCl₂FCCClF₂ and CClF₂CClF₂;
the reaction temperature is from 25° C. to less than 100° C., preferably 60° C.; and
the reaction time is from one minute to three weeks, preferably from 1 hour to 36 hours.

10. An emulsion polymerization method of forming a homopolymer from a fluoroalkene monomer according to claim 1 , which comprises polymerizing said fluoroalkene monomer in an aqueous solution at a reaction temperature with a free radical initiator, a surfactant, and optionally under a ultraviolet light;
wherein,
the free radical initiator is a water-soluble free-radical initiator selected from the group consisting of (NH₄)₂S₂O₈, Na₂S₂O₈, Li₂S₂O₈ and K₂S₂O₈, and the free-radical initiator is present at a concentration from about 0.1 mol% to 10 mol% relative to the fluoroalkene monomer;
the surfactant is a fluoro surfactant selected from the group consisting of ammonium perfluorooctanoate, sodium perfluorooctanoate, potassium perfluorooctanoate, Capstone™ surfactants, and lithium perfluorooctanoate, and the surfactant is present at a concentration from about 0.0001 wt% and 10 wt% (w/v) relative to the aqueous solution;
the reaction temperature is from 25° C. to less than 100° C., preferably 60° C.; and
the reaction time is from one minute to three weeks, preferably from 1 hour to 36 hours.

11. A method of forming a copolymer from a fluoroalkene monomer according to claim 1 , which comprises polymerizing said fluoroalkene monomer and tetrafluoroethylene in a perfluorinated solvent liquid at a reaction temperature with a perfluorinated free radical initiator, and optionally under a ultraviolet light;
wherein, the perfluorinated solvent is a fluorinated organic solvent selected from the group consisting of FC-72, HFE-7100, HFE-7500, F-626, CCl₂F₂, CCl₃F, CClF₂H, CCl₂FCCl₂F, CCl₂FCCClF₂ and CClF₂CClF₂;
the fluoroalkene monomer and tetrafluoroethylene are present at a molar ratio from 0.1 to 10;
the reaction temperature is from 25° C. to less than 100° C., preferably 60° C.; and the reaction time is from one minute to three weeks, preferably from 1 hour to 36 hours.

12. An emulsion polymerization method of forming a copolymer from a fluoroalkene monomer according to claim 1 , which comprises polymerizing said fluoroalkene monomer and tetrafluoroethylene in an aqueous solution at a reaction temperature with a free radical initiator, a surfactant, and optionally under a ultraviolet light;
wherein,
the free radical initiator is a water-soluble free-radical initiators selected from the group consisting of (NH₄)₂S₂O₈, Na₂S₂O₈, Li₂S₂O₈ and K₂S₂O₈, and the free-radical initiator is present at a concentration about from about 0.1 mol% to 10 mol% relative to the fluoroalkene monomer;
the surfactant is a fluoro surfactant selected from the group consisting of ammonium perfluorooctanoate, sodium perfluorooctanoate, potassium perfluorooctanoate, Capstone™ surfactants, and lithium perfluorooctanoate, and the surfactant is present at a concentration from about 0.0001 wt% and 10 wt% relative to the aqueous solution;
the fluoroalkene monomer and tetrafluoroethylene are present at a molar ratio from 0.1 to 10;
the reaction temperature is from 25° C. to less than 100° C., preferably 60° C.; and
the reaction time is from one minute to three weeks, preferably from 1 hour to 36 hours.

13. A proton-conductive fluoropolymer membrane comprising a structural formula of M_{z}(M^{a}ₙO₃P-PRU)ₓ(M^{b}_{n'}O₃P-R)_{y}EₘE’_{m'}•tH₂O;
wherein,
M is a metal cation selected from the group consisting of Zr, Ti, Sn, Ge, Pb, Hf, Ce, Mo and W;
M^{a} and M^{b} are independently selected from the group consisting of H, NH₄, Na, K, Cs, Li, Rb, Mg, Ca, and Sr;
n and n′ are numbers independently chosen from 0 to 2;
E and E′ are independently selected from the group consisting of OH, F, (C₁-C₆)hydrocarbon residues, and (C₁-C₆) fluorocarbon residues;
m and m′ are numbers independently chosen from 0 to 6;
t is a number chosen from 0 to 6;
•H₂O represents the structural formula comprising t number of H₂O;
z is an integer from 1, 2 or 3;
x is a number from 0.01 to 3;
y is a number from 0 to 5;
R is selected from the group consisting of H, OH, O, Cl, and a monovalent (C₁-C₁₂)hydrocarbon or fluorocarbon residue;
-PRU has a formula selected from (V)-(VIII), which is a repeating unit of a homopolymer or a copolymer: 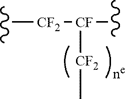 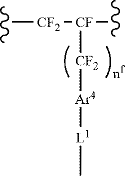 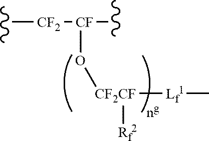 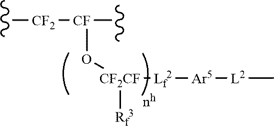
wavy lines indicate the points of attachment to adjacent repeating units of the polymer;
n^{e} and n^{f} are independent integers from 0 to 8;
n^{g} and n^{h} are independent integers from 0 to 3;
R_{f}² and R_{f}³ are independently F or CF₃;
Ar⁴ and Ar⁵ are independently (C₃-C₂₄)aromatic or heteroaromatic groups;
L¹ and L² are independently selected from the group consisting of a direct bond, and a bivalent (C₁-C₁₂)hydrocarbon or fluorocarbon residue; and
L_{f}¹ and L_{f}² are independently selected from the group consisting of a direct bond, OCF₂, OCF₂CF₂, OCF₂CF₂CF₂, OCF₂CF₂CF₂CF₂, and OCF₂CF₂CF₂CF₂CF₂.

14. A proton-conductive fluoropolymer membrane according to claim 13, wherein M is selected from the group consisting of Zr, Ti and Ce; M^{a} and M^{b} are independently selected from the group consisting of H, NH₄, Na, Li, K, Cs and Rb; n and n′ are numbers independently chosen from 0 to 2; E and E′ are independently selected from the group consisting of OH, F, (C₁-C₆) hydrocarbon residues and (C₁-C₆) fluorocarbon residues; m and m′ are numbers independently chosen from 0 to 6; t is a number from 0 to 5; z is either 1 or 2; x is from 0.01 to 3; y is from 0 to 3; R is selected from the group consisting of H, OH, O, and a monovalent (C₁-C₁₀)hydrocarbon or fluorocarbon residue; n^{e} and n^{f} are independent integers from 0 to 6; n^{g} and n^{h} are independent integers from 0 to 3; R_{f}² and Rf³ are CF₃; Ar⁴ and Ar⁵ are independently (C₃-C₁₆)aromatic or heteroaromatic groups; L¹ and L² are independently selected from the group consisting of a direct bond, and a bivalent (C₁-C₁₀)hydrocarbon or fluorocarbon residue; L_{f}¹ and L_{f}² are independently selected from a group consisting of a direct bond, OCF₂, OCF₂CF₂, OCF₂CF₂CF₂CF₂, and OCF₂CF₂CF₂CF₂CF₂.

15. A proton-conductive fluoropolymer membrane according to claim 14, wherein M is chosen from Zr or Ti; M^{a} and M^{b} are independently selected from the group consisting of H, NH₄, Na, Li, and K; n and n′ are independently chosen from 0 to 2; E and E′ are independently selected from the group consisting of OH, F, (C₁-C₆) hydrocarbon residues and (C₁-C₆) fluorocarbon residues; m and m′ are independently chosen from 0 to 4; t is from 0 to 3; z is 1; x is from 0.01 to 2; y is from 0 to 2; R is selected from the group consisting of H, OH, O, and a monovalent (C₁-C₈)hydrocarbon or fluorocarbon residue; n^{e} and n^{f} are independent from 0 to 4; n^{g} and n^{h} are independent integers from 0 to 3; Rf² and R_{f}³ are CF₃; Ar⁴ and Ar⁵ are independently (C₆-C₁₂)aromatic or heteroaromatic groups; L¹ and L² are independently selected from the group consisting of a direct bond, and a bivalent (C₁-C₆)hydrocarbon or fluorocarbon residue; and L_{f}¹ and L_{f}² are independently selected from the group consisting of OCF₂, OCF₂CF₂, OCF₂CF₂CF₂CF₂, and OCF₂CF₂CF₂CF₂CF₂.

16. A proton-conductive fluoropolymer membrane according to claim 15, wherein M is Zr; M^{a} and M^{b} are independently chosen from H or NH₄; n and n′ are from 0 to 2; E and E′ are independently selected from the group consisting of OH, F, (C₁-C₆) hydrocarbon residues; m and m′ are independently from 0 to 2; t is from 0 to 3; z is 1; x is from 0.01 to 2; y is from 0 to 2; R is selected from the group consisting of H, OH, O, and a monovalent (C₁-C₆)hydrocarbon residue; n^{e} and n^{f} are 2; n^{g} and n^{h} are independent from 0 to 2; R_{f}² and R_{f}³ are CF₃; Ar⁴ and Ar⁵ are independently (C₆-C₁₂)aromatic or heteroaromatic groups; L¹ and L² are independently selected from the group consisting of a direct bond, and a bivalent (C₁-C₆)hydrocarbon or fluorocarbon residue; and L_{f}¹ and L_{f}² are independently selected from the group consisting of OCF₂, OCF₂CF₂, OCF₂CF₂CF₂CF₂, and OCF₂CF₂CF₂CF₂CF₂.

17. A proton-conductive fluoropolymer membrane according to claim 16, wherein M is Zr; M^{a} and M^{b} are H; n and n′ are independently 0, 1 or 2; E and E′ are F; m and m′ are 0; t is either 1 or 2; z is 1; x is either 1 or 2; y is either 0 or 1; x+y=2; R is selected from the group consisting of OH, O, H, and a monovalent (C₁-C₆)hydrocarbon residue; n^{e} and n^{f} are 2; n^{g} and n^{h} are independently 0 to 2; R_{f}² and R_{f}³ are CF₃; Ar⁴ and Ar⁵ are independently (C₆-C₁₂)aromatic or heteroaromatic groups; L¹ and L² are independently selected from the group consisting of a direct bond and a bivalent (C₁-C₆)hydrocarbon or fluorocarbon residue; and L_{f}¹ and Lf² are independently selected from the group consisting of OCF₂, OCF₂CF₂, and OCF₂CF₂CF₂CF₂.

18. A proton-conductive fluoropolymer membrane according to claim 17, M is Zr; M^{a} and M^{b} are H; n is 1; n′ is 0; E and E′ are F; m and m′ are 0; t is 2; z is 1; x and y are 1; x+y=2; R is O; n^{e} and n^{f} are 2; n^{g} and n^{h} are 1; R_{f}² and R_{f}³ are CF₃; Ar⁴ and Ar⁵ are p-phenylene; L¹ and L² are CH₂; and L_{f}¹ and Lf² are OCF₂CF₂.

19. A proton-conductive fluoropolymer membrane according to claim 17, M is Zr; M^{a} and M^{b} are H; n is 1; n′ is 0; E and E′ are F; m and m′ are 0; t is 2; z is 1; y and x are 1; x+y=2; R is C₆H₄SO₃; n^{e} and n^{f} are 2; n^{g} and n^{h} are 1; R_{f}² and R_{f}³ are CF₃; Ar⁴ and Ar⁵ are p-phenylene; L¹ and L² are CH₂; and L_{f}¹ and Lf² are OCF₂CF₂.

20. A proton-conductive fluoropolymer membrane according to claim 17, wherein M is Zr; M^{a} and M^{b} are H; n is 0; n′ is 0; E and E′ are F; m is 0; m′ is 0; t is 1; z is 1; x is 2; y is 0; x+y=2; R is O; n^{e} and n^{f} are 2; n^{g} and n^{h} are 1; R_{f}² and R_{f}³ are CF₃; Ar⁴ and Ar⁵ are p-phenylene; L¹ and L² are CH₂; and L_{f}¹ and L_{f}² are OCF₂CF₂.

21. A proton-conductive composite fluoropolymer membrane comprising a proton-conductive fluoropolymer membrane according to claim 13 and optionally a second fluoropolymer selected from the group consisting of tetrafluoroethylene-perfluoro-3,6-dioxa-4-methyl-7-octenesulfonic acid copolymer, tetrafluoroethylene-perfluoro(5-oxa-6-heptenesulfonic acid) copolymer, tetrafluoroethylene-perfluoro-6,9-dioxa-5-methyl-7-undecenesulfonic acid copolymer, tetrafluoroethylene-perfluoro-6-oxa-7-octenesulfonic acid copolymer, and tetrafluoroethylene-perfluoro(3-oxa-4-pentenesulfonic acid) copolymer and poly(tetrafluoroethylene).

22. A proton-conductive composite fluoropolymer membrane according to claim 21, wherein a second fluoropolymer is selected from the group consisting of tetrafluoroethylene-perfluoro-3,6-dioxa-4-methyl-7-octenesulfonic acid copolymer, tetrafluoroethylene-perfluoro(5-oxa-6-heptenesulfonic acid) copolymer, tetrafluoroethylene-perfluoro-6,9-dioxa-5-methyl-7-undecenesulfonic acid copolymer, tetrafluoroethylene-perfluoro-6-oxa-7-octenesulfonic acid copolymer, and tetrafluoroethylene-perfluoro(3-oxa-4-pentenesulfonic acid) copolymer.

23. An impregnation method of making a proton-conductive fluoropolymer membrane, comprising
(a) eliminating the solvent from a solution comprising a fluoropolymer of perfluorophosphonic acid (PFPA);
(b) mixing the PFPA residue from (a) with a mixture comprising a metal (4+) salt having a cation selected from the group consisting of Zr⁴⁺, ZrO²⁺, Ti⁴⁺, Sn⁴⁺, Ge⁴⁺, Pb⁴⁺, Hf⁴⁺, Ce⁴⁺, Mo⁴⁺ and W⁴⁺ and an anion, and optionally a phosphonic acid or phosphoric acid in the solvent;
(c) eliminating the solvent from (b);
(d) repeating step (a), (b) and/or (c) if necessary.
wherein, the solvent of (a) is selected from the group consisting of water, phosphoric acid, hydrofluoric acid, hydrochloric acid, hydrobromic acid, hydriodic acid, sulfuric acid, nitric acid, organic solvents and combinations thereof, the PFPA is present at a concentration from 0.001 M to 50 M, preferably from 0.1 M to 1 M; and the anion of the metal (4+) salt is a negatively charged ion; in (b), the metal (4+) salt is present at a concentration from 0.001 M to 50 M, preferably from 0.1 M to 1 M; the phosphonic acid or phosphoric acid, if present, is present at a concentration from 0.001 M to 50 M, preferably from 0.1 M to 1 M; and the solvent is selected from the group consisting of water, phosphoric acid, hydrofluoric acid, hydrochloric acid, hydrobromic acid, hydriodic acid, sulfuric acid, nitric acid, organic solvents and combinations thereof.

24. An impregnation method of making a proton-conductive fluoropolymer membrane according to claim 23, wherein the metal (4+) salt has a cation of Zr⁴⁺ or ZrO²⁺; the anion of the metal (4+) salt is a negatively charged ion; the perfluorophosphonic acid (PFPA) is selected from the group consisting of tetrafluoroethylene-perfluoro-3,6-dioxa-4-methyl-7-octenephopshonic acid copolymer, perfluoro-3,6-dioxa-4-methyl-7-octenephopshonic acid homopolymer, tetrafluoroethylene-perfluoro(5-oxa-6-heptenephopshonic acid) copolymer, perfluoro-5-oxa-6-heptenephopshonic acid homopolymer, tetrafluoroethylene-perfluoro(3-oxa-4-pentenephopshonic acid) copolymer, 1,1,2,2,3,4,4-heptafluoro-3-butenylphosphonic acid homopolymer, tetrafluoroethylene-1,1,2,2,3,4,4-heptafluoro-3-butenylphosphonic acid copolymer, tetrafluoroethylene-perfluoro-6,9-dioxa-5-methyl-7-undecenephosphonic acid copolymer, tetrafluoroethylene-perfluoro-6-oxa-7-octenephosphonic acid copolymer, and perfluoro-3-oxa-4-pentenephopshonic acid homopolymer.

25. A mixing method of making a proton-conductive fluoropolymer membrane, comprising
(a) mixing a fluoropolymer of perfluorophosphonic acid (PFPA), a metal (4+) salt having a cation selected from Zr⁴⁺, Ti⁴⁺, Sn⁴⁺, Ge⁴⁺, Pb⁴⁺, Hf⁴⁺, Ce⁴⁺, Mo⁴⁺ and W⁴⁺ and an anion, and optionally a phosphonic acid or phosphoric acid in a solvent;
(b) eliminating the solvent from the mixture of (a);
(c) repeating step (a) and/or (b) if necessary.
wherein, the solvent of (a) is selected from the group consisting of water, phosphoric acid, hydrofluoric acid, hydrochloric acid, hydrobromic acid, hydriodic acid, sulfuric acid, nitric acid, organic solvents and the combination thereof; the PFPA is present at a concentration from 0.001 M to 50 M, preferably from 0.1 M to 1 M; the metal (4+) salt is present at a concentration from 0.001 M to 50 M, preferably from 0.1 M to 1 M; the anion of the metal (4+) salt is a negatively charged ion; and the phosphonic acid or phosphoric acid, if present, is present at a concentration ranges from 0.001 M to 50 M, preferably from 0.1 M to 1 M.

26. A mixing method of making a proton-conductive fluoropolymer membrane according to claim 25, wherein the metal (4+) salt has a cation of Zr⁴⁺ or ZrO²⁺; the anion of the metal (4+) salt is a negatively charged ion; the perfluorophosphonic acid (PFPA) is selected from the group consist of tetrafluoroethylene-perfluoro-3,6-dioxa-4-methyl-7-octenephopshonic acid copolymer, perfluoro-3,6-dioxa-4-methyl-7-octenephopshonic acid homopolymer tetrafluoroethylene-perfluoro(5-oxa-6-heptenephopshonic acid) copolymer, perfluoro-5-oxa-6-heptenephopshonic acid homopolymer, tetrafluoroethylene-perfluoro(3-oxa-4-pentenephopshonic acid) copolymer, 1,1,2,2,3,4,4-heptafluoro-3-butenylphosphonic acid homopolymer, tetrafluoroethylene-1,1,2,2,3,4,4-heptafluoro-3-butenylphosphonic acid copolymer, tetrafluoroethylene-perfluoro-6,9-dioxa-5-methyl-7-undecenephosphonic acid copolymer, tetrafluoroethylene-perfluoro-6-oxa-7-octenephosphonic acid copolymer, and perfluoro-3-oxa-4-pentenephopshonic acid homopolymer.

27. An electronic device selected from the group consisting of an electrolytic cell, a fuel cell, a redox flow battery, an electric storage, an electric generation device, and a water purification device, the electronic device comprising a proton-conductive fluoropolymer membrane according to claim 13 , or a proton-conductive composite fluoropolymer membrane according to claim 21 , or a fluoropolymer according to claims 7 and/or 8.
